(19) United States
(12) Patent Application Publication
(10) Pub. No.: US 2016/0030522 A1
(43) Pub. Date: Feb. 4, 2016

(54) COMBINATION THERAPY TO IMPROVE SOFT TISSUE HEALING, FAT GRAFT HEALING, ENDOCHONDRAL BONE HEALING AND OSTEOINTEGRATION

(71) Applicants: Leonard B. MILLER, Brookline, MA (US); Stephen WARREN, New York, NY (US)

(72) Inventors: Leonard B. Miller, Brookline, MA (US); Stephen Warren, New York, NY (US)

(21) Appl. No.: 14/776,854

(22) PCT Filed: Mar. 14, 2014
(86) PCT No.: PCT/US14/27549

§ 371 Date: Sep. 15, 2015

Related U.S. Application Data
(60) Provisional application No. 61/790,500, filed on Mar. 15, 2013.
(60) Provisional application No. 61/790,522, filed on Mar. 15, 2013.
(60) Provisional application No. 61/790,551, filed on Mar. 15, 2013.

(51) Int. Cl. A61K 38/29; A61K 9/00; A61K 45/06; A61K 31/395; A61K 35/35

(52) U.S. Cl. CPC A61K 38/29; A61K 31/395; A61K 35/35; A61K 45/06; A61K 9/0019

(57) ABSTRACT

The present invention is directed to kit, drug combinations and methods for promoting endogenous bone marrow (BM)-derived vasculogenic progenitor cell (PC) mobilization, sensitization of such cells and chemotaxis to the site of an injury such as injuries associated with osteointegration of implants and associated soft tissues, fat grafting and endochondral bone injuries and disease.

### RELATED APPLICATIONS

This application claims priority to U.S. provisional application Ser. No. 61/790,500 filed Mar. 15, 2013, Ser. No. 61/790,522 filed Mar. 15, 2013 and Ser. No. 61/790,551 filed Mar. 15, 2013; the entire contents of all three provisional applications are incorporated by reference herein.

### STATEMENT REGARDING FEDERAL FUNDING

Embodiments of the present invention were not conceived or reduced to practice with Federal sponsorship or funding.

### FIELD OF THE INVENTION

This application relates to a drug combinations and kits to improve soft tissue healing, fat grafting healing, endochondral bone healing and osteointegration.

### BACKGROUND OF THE INVENTION

Injury to the soft and bony tissue in healthy humans can takes months to heal. For certain procedures and surgeries which are considered elective, such long periods, often associated with substantial discomfort and lack of function, such as dental implants, can be a major factor discouraging people from otherwise beneficial operations. In certain types of implant surgeries, the implant fails to adequately integrate into the bone causing bone weakness and failure of the prosthesis. While the above treatment options can be somewhat successful, they are associated with complications such as hemarthrosis, infection, thromboembolic disease, anesthetic complications, reflex sympathetic dystrophy, iatrogenic ligament injury, iatrogenic fracture, and neurologic injuries.

Injury to the endochondral bone in healthy humans can takes months to heal. As used herein, the term “endochondral bone” refers to the long bones formed by ossification of cartilage. Injuries to such bones include, but are not limited to breaks or fractures, simple and compound, chips, and stress fractures. Current therapeutic options for the treatment of bone injuries include splints, casts, and pins and supports. While the above treatment options can be somewhat successful, they are associated with complications.

Fat grafting or fat transfer is the transplantation of fat into area of the body that requires additional volume. Areas that require additional volume are often determined for cosmetic reasons and represent patient elected surgical procedures. Typical procedures may address a patients desire to fill wrinkles associated with aging, breast augmentation, scarring from past surgical procedures, add shape or contours to facial features and other body areas. Poor healing, discomfort and periods of limited functioning or poor appearance are not readily tolerated.

Fat is typically harvested from one part of the body and injected in a desired location. This creates two areas of injury, the harvest site and the desired injection site. The harvest site may display a depression after healing due to the absence of fat. The fat injection site requires revasculization and incorporation into the surrounding tissue.

The areas of injuries limit the number and nature of the desired sites that can be addressed. The time needed for healing limits the number of procedures a patient can have and delays a meaningful evaluation of the results. Moreover, the above treatment options do not improve or augment the body's own endogenous repair mechanisms.

It is desired to have procedures, drug therapies and kits which facilitate the performance of fat grafting by promoting healing and incorporation into the tissue in which the fat is desired. To avoid the above complications and improve soft tissue healing and osteointegration of implants and improve endochondral bone healing, new therapeutic options are necessary.

### SUMMARY OF THE INVENTION

Accordingly, to overcome these challenges, the present invention encompasses, in part, promotion of endogenous bone marrow (BM)-derived vasculogenic progenitor cell (PC) mobilization, sensitization of such cells and chemotaxis to sites of injury using therapeutics or combinations of therapeutics. As used herein, the term “site of injury” refers to a place where having one or more procedures have been performed or an injury manifests itself comprising a fat grafting injury, an osteointegration site with associated soft tissues, or endochondral bone injury.

One embodiment of the present invention is directed to a method of promoting healing at one or more sites of injury. The sites of injury include an osteointegration site, such as with an implant, endochondral bone healing, fat grafting healing and closely associated soft tissue healing. The method comprises the step of administering an effective amount of a bone marrow (BM)-derived vasculogenic progenitor cell mobilization factor to an animal or human receiving the implant. The method further comprises the step of administering an effective amount of a progenitor cell sensitizing factor to mobilize progenitor cells and sensitize the progenitor cells to one or more chemotactic agents present at the site of injury.

The mobilization factor and sensitizing factor may be administered concurrently or timed for optimal effect. Concurrent administration means at or about the same time. The concurrent administration may be performed in a single occurrence or multiple occurrences over time. As used herein, the term “timed administration” refers to administration of one factor at an initial time and a second factor at an interval thereafter.

During the healing process, an adequate blood supply is critical for successful bone and closely associated soft tissue regeneration and or fat graft integration. Our recent studies have demonstrated that signals from the site of tissue injury can mobilize bone marrow (BM)-derived vasculogenic progenitor cells (PCs) into the peripheral circulation and recruit these vasculogenic PCs to the injury site where they contribute to neovascularization, tissue repair and regeneration. While we have shown that vasculogenic PC levels in the peripheral blood of humans and mice naturally increase after injury, we have also demonstrated that augmenting this natural response mechanism can dramatically improve healing. Further we have shown that small molecule-mediated mobilization of vasculogenic PCs results in increased trafficking of these PCs to the injury site, increased new blood vessel formation, and increases the speed of tissue healing.

As used herein, the term “bone marrow derived vasculogenic progenitor cell” is used as it is used in the medical and biological sciences to denote one or more stem cells which have their site of origin in the bone marrow and are released into the blood stream. This discussion will sometimes use the abbreviation “BM PC” for such term. The term “mobilization factor” is used to denote a compound or group of compounds that cause BM PCs to be released from the bone marrow into the circulation. The term “sensitizing factor” is used to denote one or more compounds which cause BM PCs to be responsive to chemotactic agents which are released by injured tissue and cause migration of BM PC to the site of injury. A chemotactic agent is a compound or group of compounds which promote the migration of BM PCs to a site of injury.

One embodiment of the present invention, directed to a method of promoting bone implant and closely associated soft tissue healing, comprises the step of administering an effective amount of a bone marrow (BM)-derived vasculogenic progenitor cell mobilization factor to an animal or human receiving the implant. The method further comprises the step of administering an effective amount of a progenitor cell sensitizing factor to mobilize progenitor cells and sensitize the progenitor cells to one or more chemotactic agents present at the site of the implant.

As used herein, the term “implant” is used to denote the a non-natural article which is placed in the bone to replace or augment the function of the body, for example, without limitation, dental implants, implants for joints such as hip, knee, ankle, foot, hand or elbow implants. Implants of solid material, such as by way of example, without limitation, metal, ceramic and plastic, are well known in the art. The term “closely associated soft tissue” is used to denote the soft tissue in proximity to the site of the implant which would and does suffer trauma as a result of the implant procedure.

One embodiment of the present method features a mobilization factor selected from the group consisting of CXCR4 agonists and partial agonists, granulocyte stimulating factor (G-CSF), granulocyte-macrophage stimulating factor (GM-CFS), interleukin-1 (Il-1), interleukin-3 (Il-3), interleukin-8 (Il-8), PIXY-321 (GM-CSF/Il-3 fusion protein), macrophage inflammatory protein, growth related oncogene and agents and factors that modify the expression of the above factors, for example without limitation, siRNA to a repressor of the above agent.

Examples of CXCR4 agonists and partial agonists are disclosed in U.S. Pat. No. 7,935,692 B2, which is incorporated by reference herein. AMD3100 is one compound which is disclosed in the '692 patent and is sold under the trademark PLERIXAFOR® (Genzyme, Boston, Mass.).

One embodiment of the present method features a sensitizing factor selected from the group consisting of parathyroid hormone and subunits of such hormone, NEL-like molecule-1, calreticulin and closely related molecules, and agents and factors that modify the expression of the above factors, such as by way of example without limitation, siRNA to a repressor of the above agent. On example of such a hormone is, without limitation, recombinant human parathyroid hormone, known as teriparatide and sold under the trademark, FORTEO®; (Eli Lilly and Company, Indianapolis, Ind.).

One embodiment of the present method features a further step of administering at least one chemotactic factor to the area of the implant. Examples of chemotactic agents include, without limitation, transforming growth factors, bone morphogenic proteins, fibroblast growth factors, vascular endothelial growth factors, stromal derived growth factors, insulin-like growth factors, nerve growth factors, myostatins, platelet derived growth factors, neurotrophins, epidermal growth factors, keratinocyte growth factors, stem cell factors, thrombopoietins, Wnt signaling proteins, hypoxia inducible factors and agents capable of modifying the expression of one or more of the above factors, such as by way of example, without limitation, siRNA directed to repressor of the above agent.

In one embodiment of the present method the mobilization factor and sensitization factor are administered by subcutaneous, intraperitoneal or intravenous injection. One embodiment features a chemotactic agent administered to the site of the implant and/or to one or more of the soft tissues proximal to the injury. The administration can be by spray, or washing with solutions loaded with such chemotactic agent or by direct injection or by coating the implant or by packing the implant with scaffolding material having a chemotactic or material which tends to be incorporated in the bone (for example bone powder) with such chemotactic agent. One embodiment of the present invention includes incorporation of the chemotactic agent into coatings on the implant. For example, without limitation, implants may comprise a porous polyethylene-polypropylene coating in which the chemotactic agent is placed. One embodiment of the present invention features incorporation of the chemotactic agent into a biopolymer which over time releases the chemotactic agent. As used herein, the term biopolymer refers to a polymer that is broken up and or consumed by the body in which it is placed by natural processes. Examples of a biopolymer include, without limitation, gelatin, polyglyconic and polylactic acid derivatives.

A further embodiment of the present invention is directed to an article of manufacture, a therapeutic dosage form comprising effective amount of a bone marrow (BM)-derived vasculogenic progenitor cell mobilization factor and an effective amount of a progenitor cell sensitizing factor to mobilize progenitor cells and sensitize the progenitor cells to one or more chemotactic agents present at the site of the implant.

One example, without limitation, of the dosage form features an effective amount of the mobilization factor and an effective amount of the sensitizing factor lyophilized and held in a vial for reconstitution, or in a vial in solution form.

A further embodiment of the dosage form comprises an effective amount of the mobilization factor and an effective amount of the sensitizing factor held in a package with an effective amount of a chemotactic agent in the form of a kit. The chemotactic agent is administered to a site of an implant or the closely associated soft tissue to direct mobilized and sensitized progenitor cells to the site where healing is desired. Preferably, the kit includes instructions and other materials and tools for making and using the elements contained therein.

For example, without limitation, the dosage form in the form of a kit may comprise a chemotactic agent lyophilized and held in a vial for reconstitution. In the event the chemotactic agent is administered by direct injection to soft tissue in and around the site of the implant, the kit may comprise an injection needle and syringe. Other embodiments feature a chemotactic agent held in coatings on the implant or a sustained release vehicle, for example, a sustained release vehicle such as a biopolymer, bone grout, bone powder and the like. Examples of biopolymers include gelatin, polyglyconic and polylactic acid derivatives. The biopolymers can be administered as microspheres or implants.

The use of a combination of mobilization factors and sensitizing factors improves healing and osteointegration of implants over healing exhibited by the use of either factor separate and apart from the other.

One embodiment of the present invention, directed to a method of promoting endochondral bone healing, comprises the step of administering an effective amount of a bone marrow (BM)-derived vasculogenic progenitor cell mobilization factor to an animal or human exhibiting bone injury or bone disease. The method further comprises the step of administering an effective amount of a progenitor cell sensitizing factor to mobilize progenitor cells and sensitize the progenitor cells to one or more chemotactic agents present at the site of bone injury or bone disease.

As used herein, the term “bone” when used as a noun, is used to denote endochondral bone unless the context of the use requires a different meaning.

The mobilization factor and sensitizing factor are as described above. The mobilization factor and sensitizing factor may be administered concurrently or timed for optimal effect. Concurrent administration means at or about the same time. The concurrent administration may be performed in a single occurrence or multiple occurrences over time. As used herein, the term “timed administration” refers to administration of one factor at an initial time and a second factor at an interval thereafter.

One embodiment of the present method features a further step of administering at least one chemotactic factor to the area of the bone injury or bone disease. Examples of chemotactic agents are as described above. The detailed discussion that follows features the stromal derived growth factor, stromal cell derived factor-1 (SDF-1).

In one embodiment of the present method the mobilization factor and sensitization factor are administered by subcutaneous, intraperitoneal or intravenous injection. However, other modes of administration may be used including by way of example, without limitation, oral, sublingual, buccal, rectal, nasal, transdermal and pulmonary administration.

One embodiment features a chemotactic agent is administered to the site of injury or to the site of bone disease to one or more of the soft tissues proximal to the injury. The administration can be by spray, or washing with solutions loaded with such chemotactic agent or by direct injection. One embodiment of the present invention features incorporation of the chemotactic agent into a biopolymer which over time releases the chemotactic agent. As used herein, the term biopolymer refers to a polymer that is broken up and or consumed by the body in which it is placed by natural processes. Examples of a biopolymer include, without limitation, gelatin, polyglyconic and polylactic acid derivatives.

A further embodiment of the present invention is directed to an article of manufacture, a therapeutic dosage form comprising effective amount of a bone marrow (BM)-derived vasculogenic progenitor cell mobilization factor and an effective amount of a progenitor cell sensitizing factor to mobilize progenitor cells and sensitize the progenitor cells to one or more chemotactic agents present at the site of bone injury or bone disease.

One example, without limitation, of the dosage form features an effective amount of the mobilization factor and an effective amount of the sensitizing factor lyophilized and held in a vial for reconstitution, or in a vial in solution form.

A further embodiment of the dosage form comprises an effective amount of the mobilization factor and an effective amount of the sensitizing factor held in a package with an effective amount of a chemotactic agent in the form of a kit. The chemotactic agent is administered to a disease bone or an injured bone to direct mobilized and sensitized progenitor cells to the site where healing is desired. Preferably, the kit includes instructions and other materials and tools for making and using the elements contained therein.

For example, without limitation, the dosage form in the form of a kit may comprise a chemotactic agent lyophilized and held in a vial for reconstitution. In the event the chemotactic agent is administered by direct injection to soft tissue in and around the injured bone or diseased bone, the kit may comprise an injection needle and syringe. Other embodiments feature a chemotactic agent held in a sustained release vehicle, for example, a sustained release vehicle such as a biopolymer. Examples of biopolymers include gelatin, polyglyconic and polylactic acid derivatives. The biopolymers can be administered as microspheres or implants.

The use of a combination of mobilization factors and sensitizing factors improves healing of bone injuries and bone disease over healing exhibited by the use of either factor separate and apart from the other.

One embodiment of the present invention, directed to a method of promoting fat grafting and fat harvesting healing, comprises the step of administering an effective amount of a bone marrow (BM)-derived vasculogenic progenitor cell mobilization factor to an animal or human having a site in which at least one procedure selected from the group consisting of fat grafting and fat harvesting will be performed. The method further comprises the step of administering an effective amount of a progenitor cell sensitizing factor to mobilize progenitor cells and sensitize the progenitor cells to one or more chemotactic agents present at the site of the fat harvesting and/or graft.

As used herein, the term “fat” is used to denote adipose tissue cells. Fat grafting is the transplantation of adipose tissue cells from one place of an individual or animal into another place; or the transplantation of adipose tissue cells from one individual or animal into a second individual or animal. Cosmetic fat grafting procedures are typically performed in which fat is harvested from one part of the body and relocated into another part of the body.

The mobilization factor and sensitizing factor are as described above. The mobilization factor and sensitizing factor may be administered concurrently or timed for optimal effect. Concurrent administration means at or about the same time. The concurrent administration may be performed in a single occurrence or multiple occurrences over time. As used herein, the term “timed administration” refers to administration of one factor at an initial time and a second factor at an interval thereafter.

One embodiment of the present method features a further step of administering at least one chemotactic factor to the area of the fat harvesting or fat grafting. Examples of chemotactic factors have been described above. The detailed discussion that follows features the stromal derived growth factor, stromal cell derived factor-1 (SDF-1),

In one embodiment of the present method the mobilization factor and sensitization factor are co-administered by subcutaneous, intraperitoneal or intravenous injection. However other routes of administration can be readily used, including, by way of example, without limitation, oral, sublingual, buccal, nasal, rectal, nasal, pulmonary, and transdermal administration.

One embodiment features a chemotactic agent administered to the site of fat grafting and/or fat harvesting or to one or more of the soft tissues proximal to the injury. The administration can be by spray, or washing with solutions loaded with such chemotactic agent or by direct injection or co-administration with the fat cells. One embodiment of the present invention features incorporation of the chemotactic agent into a biopolymer which over time releases the chemotactic agent. As used herein, the term biopolymer refers to a polymer that is broken up and or consumed by the body in which it is placed by natural processes. Examples of a biopolymer include, without limitation, gelatin, polyglyconic and polylactic acid derivatives. The chemotactic agent can be co-administered by combining the agent with fat cells prior to placing the fat cells into the desired site.

A further embodiment of the present invention is directed to an article of manufacture, a therapeutic dosage form comprising effective amount of a bone marrow (BM)-derived vasculogenic progenitor cell mobilization factor and an effective amount of a progenitor cell sensitizing factor to mobilize progenitor cells and sensitize the progenitor cells to one or more chemotactic agents present at one or more sites selected from the group consisting of fat grafting and fat harvesting.

One example, without limitation, of the dosage form features an effective amount of the mobilization factor and an effective amount of the sensitizing factor lyophilized and held in one or more vials for reconstitution, or in one or more vials in solution form.

A further embodiment of the dosage form comprises an effective amount of the mobilization factor and an effective amount of the sensitizing factor held in a package with an effective amount of a chemotactic agent in the form of a kit. The chemotactic agent is administered to at least one site selected from the group consisting of fat grafting and fat harvesting to direct mobilized and sensitized progenitor cells to the site where healing is desired. Preferably, the kit includes instructions and other materials and tools for making and using the elements contained therein.

For example, without limitation, the dosage form in the form of a kit may comprise a chemotactic agent lyophilized and held in a vial for reconstitution. In the event the chemotactic agent is administered by direct injection to soft tissue in and around the site of the graft or harvesting, the kit may comprise an injection needle and syringe. Other embodiments feature a chemotactic agent held in a sustained release vehicle, for example, a sustained release vehicle such as a biopolymer. Examples of biopolymers include gelatin, polyglyconic and polylactic acid derivatives. The biopolymers can be administered as microspheres or implants. Another embodiment features a chemotactic agent co-administered with adipose cells to be grafted.

The use of a combination of mobilization factors and sensitizing factors improves healing of sites of fat grafting and fat harvesting over healing exhibited by the use of either factor separate and apart from the other.

Other features, objects, and advantages of the invention will be apparent to those skilled in the art upon viewing the drawings which are described briefly below and reading the detailed description that follows. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs.

### BRIEF DESCRIPTION OF THE DRAWING

FIG. 1 depicts a dosage form incorporated into a kit embodying features of the present invention.

### DETAILED DESCRIPTION OF THE INVENTION

Embodiment of the present invention will now be described in detail with respect to an article of manufacture, a therapeutic dosage form reflecting the preferred embodiments of the invention. Those skilled in the art will recognize that the details of such dosage form can be modified and altered and what is thought to be preferred embodiments may change over time. Therefore the present discussion should not be considered limiting.

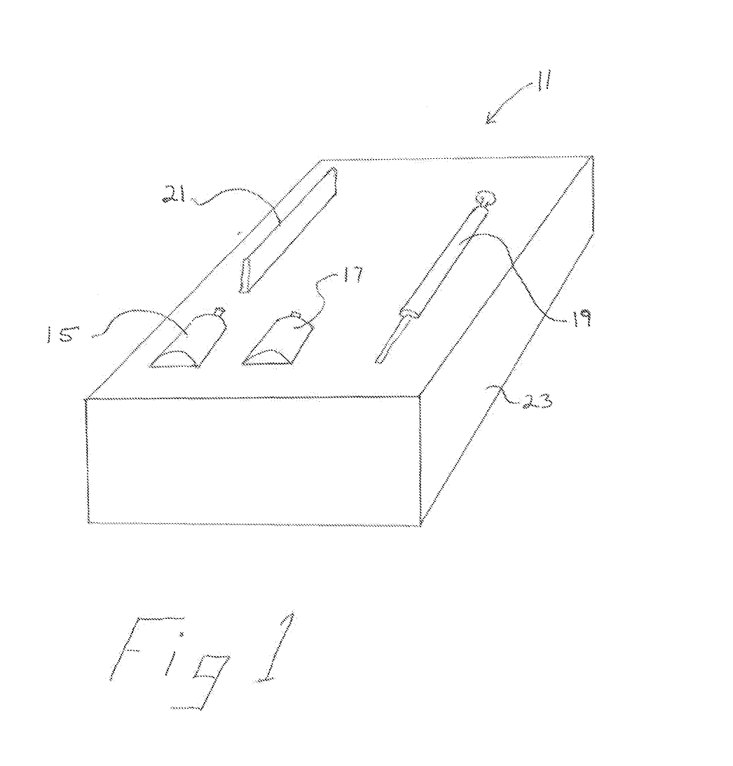

Turning now to FIG. 1, a kit embodying features of the present invention, generally designated by the numeral 11, is depicted. The kit 11 is for promoting soft tissue healing and osteointegration of an implant, or for promoting endochondral bone healing, or for promoting fat grafting healing.

The kit has the following major components, a first vial 15, a second vial 17, a syringe 19, instruction for use 21 and packaging in the form of a box 23. Although a box 23 is depicted, suitable packaging may take many forms. For example, without limitation, suitable packaging may comprise bags, plastic or paper wraps, bundles and the like known in the art. The box 23 is preferably fitted with a cover [not shown] to provide a more complete contained enclosure.

First vial 15 containing at least two compounds, bone marrow (BM)-derived vasculogenic progenitor cell mobilization factor and progenitor cell sensitizing factor. The mobilization factor is selected from the group consisting of CXCR4 agonists and partial agonists, granulocyte stimulating factor (G-CSF), granulocyte-macrophage stimulating factor (GM-CFS), interleukin-1 (Il-1), interleukin-3 (Il-3), interleukin-8 (Il-8), PIXY-321 (GM-CSF/Il-3 fusion protein), macrophage inflammatory protein, and growth related oncogene and agents and factors that modify the expression of the above factors, for example without limitation, siRNA to a repressor of the above agent. For the purpose of this discussion, the mobilization factor is a CXCR4 partial agonist, AMD3100, disclosed in the '692 patent and is sold under the trademark PLERIXAFOR® (Genzyme, Boston, Mass.).

The sensitizing factor is selected from the group consisting of parathyroid hormone and subunits of such hormone, NEL-like molecule-1, calreticulin and closely related molecules, and agents and factors that modify the expression of the above factors such as by way of example without limitation, siRNA to a repressor of the above agent. For the purpose of this discussion, the sensitizing factor is recombinant human parathyroid hormone, known as teriparatide and sold under the trademark, FORTEO® (Eli Lilly and Company, Indianapolis, Ind.).

The two compounds are held as lyophilized powders for reconstitution in first vial 15. Upon reconstitution, the powders form a solution for injection in which an injection will administer AMD3100 (approximately 8-12 mg/kg of weight of individual or animal) and teriparatide (approximately 0.228-0.342 mcg/kg of weight of individual or animal). These amounts represent an effective amount of a bone marrow (BM)-derived vasculogenic progenitor cell mobilization factor and an effective amount of a progenitor cell sensitizing factor to mobilize progenitor cells and sensitize the progenitor cells to one or more chemotactic agents present at the site of the implant. In the alternative, the mobilization factor and the sensitizing factor are held in separate vials and administered separately in a timed sequential manner or simultaneously.

These effective amounts of bone marrow (BM)-derived vasculogenic progenitor cell mobilization factor and progenitor cell sensitizing factor are administered to the individual or animal by subcutaneous, intraperitoneal, intramuscular injection by syringe 19. However, other means for providing concurrent administration of the mobilization factor and sensitizing factor may be used including, by way of example, without limitation, oral, sublingual, buccal, nasal, pulmonary, rectal, transdermal and ocular administration.

The second vial 17 containing a chemotactic agent lyophilized for reconstitution. Examples of chemotactic agents include, without limitation, transforming growth factors, bone morphogenic proteins, fibroblast growth factors, vascular endothelial growth factors, stromal derived growth factors, insulin-like growth factors, nerve growth factors, myostatins, platelet derived growth factors, neurotrophins, epidermal growth factors, keratinocyte growth factors, stem cell factors, thrombopoietins, Wnt signaling proteins, hypoxia inducible factors and agents capable of modifying the expression of one or more of the above factors, such as by way of example, without limitation, siRNA directed to repressor of the above agent. For the purpose of this discussion, the chemotactic agent is stromal cell derived factor-1 (SDF-1).

SDF-1 is administered in an amount ranging from 1.00 ng to about 100 ng. In the event the chemotactic agent is administered by direct injection to soft tissue closely associated with the implant, the kit 11 may comprise a second injection needle and syringe [not shown]. Other embodiments feature a chemotactic agent held or placed in porous coating of the implant or in a sustained release vehicle, for example, a sustained release vehicle such as a biopolymer. Examples of biopolymers include gelatin, polyglyconic and polylactic acid derivatives. The biopolymers can be administered as microspheres or implants. The chemotactic agent is administered to a site of an implant to direct mobilized and sensitized progenitor cells to the site where healing is desired.

The kit 11 includes instructions 21 and other materials and tools for making and using the elements contained therein. For example, the kit 11 can comprise a device for implantation [not shown]. The instructions 21 will be described now in relationship to the method of using the kit 11.

The instructions 21 set forth a method of promoting soft tissue healing and osteointegration of an implant, or for promoting endochondral bone healing, or for promoting fat grafting healing.

The method comprises the step of administering an effective amount of a bone marrow (BM)-derived vasculogenic progenitor cell mobilization factor to an animal or human receiving an implant, having a bone injury or fat graft. And, the method comprises the step of administering, concurrently to the mobilization factor, an effective amount of a progenitor cell sensitizing factor. The mobilization factor and sensitizing factor are reconstituted from the compounds in the first vial 15 and withdrawn from the first vial 15 with syringe 19. Syringe 19 is used to inject an effective amount of the mobilization factor and sensitizing factor subcutaneously, intraperitoneal, or intramuscularly into individual or animal to mobilize progenitor cells and sensitize the progenitor cells to one or more chemotactic agents present at the site of the implant, bone injury or fat graft injury.

The chemotactic agent is reconstituted from the powder held in second vial 17 and administered to the site of the implant and/or soft tissue closely associated with the implant. The site may have naturally occurring chemotactic agents and make the administration of the reconstituted chemotactic agent optional. Where the site of injury is a fat grafting site, the chemotactic agent is administered by combining with the fat cells forming the graft.

The use of a combination of mobilization factors and sensitizing factors improves healing of implants, bone injury and/or fat graft site over healing exhibited by the use of either factor separate and apart from the other.

### Example #1

### Implant

Mice and Injury Model: All experiments are performed in accordance with the IACUC guidelines. C57BL/6J wild-type mice aged 8-12 weeks are purchased from Jackson Laboratories (Bar Harbor, Me.). Mice are randomized to receive one of #1) no injury; #2) an implant stud having a porous polyethylene-polypropylene coating placed in a defect in the distal femur. This injury models resemble the bone implant common in humans.

Treatment Groups: Mice in each of the 4 experimental groups are randomly assigned to receive once daily one of: #1) saline i.p. injection; #2) AMD3100 (10 mg/kg, i.p.; PLERIXAFOR®; Genzyme Corp., Cambridge, Mass.) injection; #3) Teriparatide (0.285 mcg/kg, i.p.; FORTEO®; Eli Lilly and Company, Indianapolis, Ind.); or #4) AMD3100 (10 mg/kg, i.p.; PLERIXAFOR®; Genzyme Corp., Cambridge, Mass.); and teriparatide (0.285 mcg/kg, i.p.; FORTEO®; Eli Lilly and Company, Indianapolis, Ind.).

Further experimental groups can be made with mice randomly assigned to receive one dose of SDF-1 is administered in an amount ranging from 1.00 ng to about 100 ng or one dose of saline by direct injection to soft tissue closely associated with the implant, and one dose of SDF-1 in an amount of 1.00 ng to about 100 ng in the porous coating of the implant stud.

Isolation of Mononuclear Cells (MNCs) from Peripheral Blood and Bone Marrow: Peripheral blood (PB) is harvested from mice at baseline, 7, 14, and 21 days post-wounding 1-hour following treatment with AMD3100, PTH, AMD3100+PTH, or sterile saline. BM is flushed from mouse long bones using PBS/10% FBS/5% EDTA, as previously described. Mononuclear cells (MNCs) from the peripheral blood and BM are isolated by density gradient centrifugation using Histopaque 1083 (Sigma-Aldrich; St. Louis, Mo.).

Flow Cytometry and Isolation of Progenitor Cells: For characterization by flow cytometry, PB MNCs are labeled with rat anti-mouse antibodies (fluorescein isothiocyanate conjugated Sca-1, allophycocyanin-conjugated c-kit, strepavidin-PE-conjugated-Cy7)(BD Bioscience; San Jose, Calif. and Miltenyi Biotech). All antibodies are titrated and optimized for appropriate detection. Samples are collected using a BD FACSCaliber flow cytometer (Becton-Dickinson; Franklin Lakes, N.J.) and analyses are performed with FlowJo 8.0 software (TreeStar Inc.; Ashland, Oreg.).

PCs are isolated from BM-MNCs by magnetic cell separation using a commercially available mouse lineage depletion kit (MACS®, Miltenyi Biotec, Inc.; Auburn, Calif.). Using this kit, lineage positive cells are removed, leaving an enriched lineage negative (lin−) cell population.

Isolated lin− cells are stained with FITC-Sca-1, APC-c-kit and sorted using a Dako MoFlo cell sorter (Dako Colorado Inc.; Fort Collins, Colo.). Enriched lin−/Sca-1+/c-kit+ cells (L−S+C+) are seeded onto 24-well plates (1,000 cells/well) (Corning Costar, Lowell, Mass.) and expanded in StemSpan Serum-Free media (Stem Cell Technologies; Vancouver, BC, Canada) supplemented with thrombopoietin [TPO: 20 ng/mL], stem cell factor [SCF: 100 ng/mL], interleukin-6 [IL-6:20 ng/mL], vascular endothelial growth factor [VEGF: 50 ng/mL], and Flt-3 [100 ng/mL] (Peprotech; Rocky Hill, N.J.). The L−S+C+ cell population is heterogenous, but enriched for vasculogenic PCs (Tepper O M, Carr J, Allen R J, Jr., Chang C C, Lin C D, Tanaka R, Gupta S M, Levine J P, Saadeh P B, Warren S M: Decreased circulating progenitor cell number and failed mechanisms of stromal cell-derived factor-1 alpha mediated bone marrow mobilization impair diabetic tissue repair. Diabetes 2010; 59:1974-1983, the contents of which are hereby incorporated by reference in its entirety). Supplemented StemSpan is considered vasculogenic PC growth medium. All assays are performed on primary cultured PCs following 7 days of expansion.

Chemotaxis Assay: PC migration is measured using a modified Boyden chamber assay as previously described. Briefly, SDF-1α (100 ng/mL), PDGF-BB (100 ng/mL) or FBS (control) in vasculogenic PC growth medium or standard cell growth media is placed in the bottom of a 24-well plate. Cells (5×104)±AMD3100 (5-50 ng/mL)±rhPTH (5-50 ng/mL) is seeded onto fibronectin-coated (5 μg/cm2) transwell inserts. After 20 hours cells are harvested from the bottom chambers, washed, and centrifuged. Cell pellets are frozen at −80° C. Frozen cells are resuspended in CyQuant Green Fluorescent dye (Invitrogen) and the relative fluorescence is measured using a Synergy™ HT microplate reader (BioTek; Winooski, Vt.).

Adhesion Assay: Adhesion of PCs is measured in AMD3100 (5-50 ng/mL)±rhPTH (5-50 ng/mL). PCs (1×105 cells/chamber) are added to 4 well chamber slides (Fisher Scientific; Pittsburgh, Pa.) coated with fibronectin (5 μg/cm2) (Sigma) and incubated at 37° C. for 2 hours. Following incubation, non-adherent cells are removed before adherent cells are fixed with 1% paraformaldehyde. Adherent cells are stained with DAPI (4′,6 diamidino-2-phenylindole) (VectaShield; Vector Laboratories, Burlingame, Calif.) and viewed on an Olympus BX51 epifluorescent microscope.

Adobe Photoshop CS3 (Adobe Systems; San Jose, Calif.) is used to quantify the number of cells/random high-powered field (hpf) under 100× magnification.

Proliferation Assay: Proliferation of PCs is measured using BrdU (5-Bromo-2′ deoxyuridine) labeling and fluorescent detection (Synergy™ HT microplate reader: BioTek; Winooski, Vt.). Proliferation is compared in media containing AMD3100 (5-50 ng/mL)±rhPTH (5-50 ng/mL).

Histology and Immunofluorescence: Bone is harvested on days 14, 21, and 28 for analysis. Frozen sections are stained with rat anti-mouse CD31 (PECAM; BD Biosciences) primary antibody and goat anti-rat IgG secondary (Alexafluor 594; Invitrogen). Control samples are prepared without primary antibody. Slides are mounted with DAPI (Sigma) and viewed on an Olympus BX51 epifluorescent microscope. DAPI is used to determine the sample outline; whereas, immunofluorescent CD31 staining is used to identify vascular structures (red staining) within the sample. Dual filter images are superimposed to illustrate wound architecture and vascular staining. Adobe Photoshop CS3 is used to segment and quantify positive CD31 staining. The vascular density of mouse wounds is determined by quantifying the total area of CD31+ staining (red) per megapixel (1×106 pixels square area) of wound stained. Paraffin sections are stained with hematoxylin and eosin (H&E) to compare wound architecture between treatment groups as well as to confirm the full-thickness nature of the punch biopsies.

Statistical Analysis: Data is presented as mean standard error of the mean. A one way ANOVA with post-hoc Tukey Kramer is used for comparison of wound closure rates, cPC number, and vascular staining between all groups studied. A Student's t test is used for comparison between groups for the functional assays. Statistical significance is considered to be p<0.05. The number of mice per treatment group is determined using G*Power (G*Power®, Melbourne, Australia) to provide a power greater than 0.80.

### Discussion

Our evidence suggests that vascularization plays a significant role in tissue healing. The present application, encompasses, in part, an endogenous strategy to improve bone and soft tissue healing by promoting revascularization. In the present study, we show that endogenously mobilizing stem cells and concomitantly enhancing their trafficking yields a remarkable increase in healing. While systemic AMD3100 administration resulted in 59.7% bony ingrowth and PTH alone resulted in 56% bony ingrowth, together a synergistic effect of 90.6% bony regeneration was achieved; this was associated with significantly increased numbers of cPCs and CD31 staining in the trephine defect. Our results suggest that mobilized vasculogenic PCs increase new blood vessel formation at the site of injury and substantially increase bony regeneration. Since the effect of combining AMD3100 and rhPTH was synergistic, it was clear that rhPTH was not just acting through a local proliferative osteoprogenitor effect, but was effectively improving PC trafficking. Our in vitro adhesion assay results strongly support that the nature of systemic rhPTH synergistic effect was through improved cPCs trafficking and tubule formation.

### Examples #2

### Endochondral Bone

Mice and Injury Model: All experiments are performed in accordance with the IACUC guidelines. C57BL/6J wild-type mice aged 8-12 weeks are purchased from Jackson Laboratories (Bar Harbor, Me.). Mice are randomized to receive one of #1) no injury; #2) distal femoral fracture. These injury models resemble a bone injury common in humans.

Treatment Groups: Mice in each of the 4 experimental groups are randomly assigned to receive once daily one of: #1) saline i.p. injection; #2) AMD3100 (10 mg/kg, i.p.; PLERIXAFOR®; Genzyme Corp., Cambridge, Mass.) injection; #3) Teriparatide (0.285 mcg/kg, i.p.; FORTEO®; Eli Lilly and Company, Indianapolis, Ind.); or #4) AMD3100 (10 mg/kg, i.p.; PLERIXAFOR®; Genzyme Corp., Cambridge, Mass.); and teriparatide (0.285 mcg/kg, i.p.; FORTEO®; Eli Lilly and Company, Indianapolis, Ind.).

Further experimental groups can be made with mice randomly assigned to receive one dose of SDF-1 is administered in an amount ranging from 1.00 ng to about 100 ng or one dose of saline by direct injection to soft tissue in and around the injured bone.

Isolation of Mononuclear Cells (MNCs) from Peripheral Blood and Bone Marrow: Peripheral blood (PB) is harvested from mice at baseline, 7, 14, and 21 days post-wounding 1-hour following treatment with AMD3100, PTH, AMD3100+PTH, or sterile saline. BM is flushed from mouse long bones using PBS/10% FBS/5% EDTA, as previously described. Mononuclear cells (MNCs) from the peripheral blood and BM are isolated by density gradient centrifugation using Histopaque 1083 (Sigma-Aldrich; St. Louis, Mo.).

Flow Cytometry and Isolation of Progenitor Cells: For characterization by flow cytometry, PB MNCs are labeled with rat anti-mouse antibodies (fluorescein isothiocyanate conjugated Sca-1, allophycocyanin-conjugated c-kit, strepavidin-PE-conjugated-Cy7)(BD Bioscience; San Jose, Calif. and Miltenyi Biotech). All antibodies are titrated and optimized for appropriate detection. Samples are collected using a BD FACSCaliber flow cytometer (Becton-Dickinson; Franklin Lakes, N.J.) and analyses are performed with FlowJo 8.0 software (TreeStar Inc.; Ashland, Oreg.).

PCs are isolated from BM-MNCs by magnetic cell separation using a commercially available mouse lineage depletion kit (MACS®, Miltenyi Biotec, Inc.; Auburn, Calif.). Using this kit, lineage positive cells are removed, leaving an enriched lineage negative (lin−) cell population.

Isolated lin− cells are stained with FITC-Sca-1, APC-c-kit and sorted using a Dako MoFlo cell sorter (Dako Colorado Inc.; Fort Collins, Colo.). Enriched lin−/Sca-1+/c-kit+ cells (L−S+C+) are seeded onto 24-well plates (1,000 cells/well) (Corning Costar, Lowell, Mass.) and expanded in StemSpan Serum-Free media (Stem Cell Technologies; Vancouver, BC, Canada) supplemented with thrombopoietin [TPO: 20 ng/mL], stem cell factor [SCF: 100 ng/mL], interleukin-6 [IL-6:20 ng/mL], vascular endothelial growth factor [VEGF: 50 ng/mL], and Flt-3 [100 ng/mL] (Peprotech; Rocky Hill, N.J.). The L−S+C+ cell population is heterogenous, but enriched for vasculogenic PCs (Tepper O M, Carr J, Allen R J, Jr., Chang C C, Lin C D, Tanaka R, Gupta S M, Levine J P, Saadeh P B, Warren S M: Decreased circulating progenitor cell number and failed mechanisms of stromal cell-derived factor-1 alpha mediated bone marrow mobilization impair diabetic tissue repair. Diabetes 2010; 59:1974-1983, the contents of which are hereby incorporated by reference in its entirety). Supplemented StemSpan is considered vasculogenic PC growth medium. All assays are performed on primary cultured PCs following 7 days of expansion.

Chemotaxis Assay: PC migration is measured using a modified Boyden chamber assay as previously described. Briefly, SDF-1α (100 ng/mL), PDGF-BB (100 ng/mL) or FBS (control) in vasculogenic PC growth medium or standard cell growth media is placed in the bottom of a 24-well plate. Cells (5×104)±AMD3100 (5-50 ng/mL)±rhPTH (5-50 ng/mL) is seeded onto fibronectin-coated (5 μg/cm2) transwell inserts. After 20 hours cells are harvested from the bottom chambers, washed, and centrifuged. Cell pellets are frozen at −80° C. Frozen cells are resuspended in CyQuant Green Fluorescent dye (Invitrogen) and the relative fluorescence is measured using a Synergy™ HT microplate reader (BioTek; Winooski, Vt.).

Adhesion Assay: Adhesion of PCs is measured in AMD3100 (5-50 ng/mL)±rhPTH (5-50 ng/mL). PCs (1×105 cells/chamber) are added to 4 well chamber slides (Fisher Scientific; Pittsburgh, Pa.) coated with fibronectin (5 μg/cm2) (Sigma) and incubated at 37° C. for 2 hours. Following incubation, non-adherent cells are removed before adherent cells are fixed with 1% paraformaldehyde. Adherent cells are stained with DAPI (4′,6 diamidino-2-phenylindole) (VectaShield; Vector Laboratories, Burlingame, Calif.) and viewed on an Olympus BX51 epifluorescent microscope. Adobe Photoshop CS3 (Adobe Systems; San Jose, Calif.) is used to quantify the number of cells/random high-powered field (hpf) under 100× magnification.

Proliferation Assay: Proliferation of PCs is measured using BrdU (5-Bromo-2′ deoxyuridine) labeling and fluorescent detection (Synergy™ HT microplate reader: BioTek; Winooski, Vt.). Proliferation is compared in media containing AMD3100 (5-50 ng/mL)±rhPTH (5-50 ng/mL).

Histology and Immunofluorescence: Bone is harvested on days 14, 21, and 28 for analysis. Frozen sections are stained with rat anti-mouse CD31 (PECAM; BD Biosciences) primary antibody and goat anti-rat IgG secondary (Alexafluor 594; Invitrogen). Control samples are prepared without primary antibody. Slides are mounted with DAPI (Sigma) and viewed on an Olympus BX51 epifluorescent microscope. DAPI is used to determine the sample outline; whereas, immunofluorescent CD31 staining is used to identify vascular structures (red staining) within the sample. Dual filter images are superimposed to illustrate wound architecture and vascular staining. Adobe Photoshop CS3 is used to segment and quantify positive CD31 staining. The vascular density of mouse wounds is determined by quantifying the total area of CD31+ staining (red) per megapixel (1×106 pixels square area) of wound stained. Paraffin sections are stained with hematoxylin and eosin (H&E) to compare wound architecture between treatment groups as well as to confirm the full-thickness nature of the punch biopsies.

Statistical Analysis: Data is presented as mean standard error of the mean. A one way ANOVA with post-hoc Tukey Kramer is used for comparison of wound closure rates, cPC number, and vascular staining between all groups studied. A Student's t test is used for comparison between groups for the functional assays. Statistical significance is considered to be p<0.05. The number of mice per treatment group is determined using G*Power (G*Power®, Melbourne, Australia) to provide a power greater than 0.80.

### Discussion

Our evidence suggests that vascularization plays a significant role in tissue healing. The present application, encompasses, in part, an endogenous strategy to improve bone healing by promoting revascularization. In the present study, we show that endogenously mobilizing stem cells and concomitantly enhancing their trafficking yields a remarkable increase in bony healing. While systemic AMD3100 administration resulted in 59.7% bony ingrowth and PTH alone resulted in 56% bony ingrowth, together a synergistic effect of 90.6% bony regeneration was achieved; this was associated with significantly increased numbers of cPCs and CD31 staining in the trephine defect. Our results suggest that mobilized vasculogenic PCs increase new blood vessel formation at the site of injury and substantially increase bony regeneration. Since the effect of combining AMD3100 and rhPTH was synergistic, it was clear that rhPTH was not just acting through a local proliferative osteoprogenitor effect, but was effectively improving PC trafficking. Our in vitro adhesion assay results strongly support that the nature of systemic rhPTH synergistic effect was through improved cPCs trafficking and tubule formation.

These findings reinforce that tissue healing is multifactorial. The present inventors have found that the combination of teriparatide (1-34 portion of PTH) and AMD3100 (PLERIXAFOR®) will enhance endochondral bone healing. Without wishing to be bound by theory, targeting two completely different pathways, both equally essential to bone and soft tissue growth, we will provide a level of healing not demonstrated before.

### Example #3

### Fat Grafting

Harvest of Human Fat: Human adipose tissue will be lipoaspirated from the abdomen or thighs and centrifuged at 1,188 g centrifugal force for 3 minutes. Following centrifugation, the blood/tumescent fraction will be drained and the oil removed. 1.0 cc of the highest density (HD), 1.0 cc of the lowest density (LD), and 1.0 cc of mixed density (MD) lipoaspirate will then used for grafting experiments.

Mice and Fat Grafting Model: All experiments are performed in accordance with the IACUC guidelines. A previously described, 8-week old male FVB mouse (Jackson Laboratory; Bar Harbor, Me.) fat grafting model was used. A small (˜2 mm) access incision will be made at the root of the tail and exactly 2 cc of HD, LD, MD fat will be injected (˜0.03 cc/pass) in a fan-like pattern to evenly layer the fat in the dorsal subcutaneous tissues superficial to the muscular panniculus carnosus using a 17-gauge cannula (Mentor Corporation, Santa Monica, Calif.).

Treatment Groups: Immediately after grafting and continuing for 14 days, mice (sample size to be determined) that received HD, LD, MD will be injected with: 1) normal saline s.c.; 2) AMD3100 (10 mg/kg i.p./day; Sigma-Aldrich; St. Louis, Mo.); 3) rhPTH (5 μg/kg, s.c.; GenScript; Piscataway, N.J.); 4) AMD3100 (10 mg/kg i.p./day; Sigma-Aldrich; St. Louis, Mo.) and rhPTH (5 μg/kg, s.c.; GenScript; Piscataway, N.J.).

Further experimental groups can be made with mice randomly assigned to receive one dose of SDF-1 is administered in an amount ranging from 1.00 ng to about 100 ng or one dose of saline by direct injection to soft tissue in and around the fat harvesting area or the fat grafting area.

Fat Graft Survival Assessment: At 3 months, a dorsal-lateral incision will be made and the skin was reflected to reveal the underlying human fat. Injected fat will be harvested en bloc using a stereoscopic dissecting microscope (Stemi SV11, Carl Zeiss, Inc, Thornwood, N.Y.) and immediately weighed on a dual range analytical balance (Mettler AE 240, International Equipment Trading Ltd, Vernon Hills, Ill.). The percentage fat graft survival will be determined as a weight/weight (w/w) ratio of initial weight (grams) to the final weight (grams) as expressed in the following formula:

Fat graft Survival (%)=[initial weight(grams)/final weight(grams)]×100.

Histologic, Protein, and Nucleic Acid Analysis: At 3 months, immunohistologic, protein, and RNA analyses will be performed to assay factors known to be important in fat graft survival.

Isolation of Mononuclear Cells (MNCs) from Peripheral Blood and Bone Marrow: Peripheral blood (PB) is harvested from mice at baseline, 7, 14, and 21 days post-wounding 1-hour following treatment with AMD3100, PTH, AMD3100+PTH, or sterile saline. BM is flushed from mouse long bones using PBS/10% FBS/5% EDTA, as previously described. Mononuclear cells (MNCs) from the peripheral blood and BM are isolated by density gradient centrifugation using Histopaque 1083 (Sigma-Aldrich; St. Louis, Mo.).

Flow Cytometry and Isolation of Progenitor Cells: For characterization by flow cytometry, PB MNCs are labeled with rat anti-mouse antibodies (fluorescein isothiocyanate conjugated Sca-1, allophycocyanin-conjugated c-kit, strepavidin-PE-conjugated-Cy7)(BD Bioscience; San Jose, Calif. and Miltenyi Biotech). All antibodies are titrated and optimized for appropriate detection. Samples are collected using a BD FACSCaliber flow cytometer (Becton-Dickinson; Franklin Lakes, N.J.) and analyses are performed with FlowJo 8.0 software (TreeStar Inc.; Ashland, Oreg.).

PCs are isolated from BM-MNCs by magnetic cell separation using a commercially available mouse lineage depletion kit (MACS®, Miltenyi Biotec, Inc.; Auburn, Calif.). Using this kit, lineage positive cells are removed, leaving an enriched lineage negative (lin−) cell population.

Isolated lin− cells are stained with FITC-Sca-1, APC-c-kit and sorted using a Dako MoFlo cell sorter (Dako Colorado Inc.; Fort Collins, Colo.). Enriched lin−/Sca-1+/c-kit+ cells (L−S+C+) are seeded onto 24-well plates (1,000 cells/well) (Corning Costar, Lowell, Mass.) and expanded in StemSpan Serum-Free media (Stem Cell Technologies; Vancouver, BC, Canada) supplemented with thrombopoietin [TPO: 20 ng/mL], stem cell factor [SCF: 100 ng/mL], interleukin-6 [IL-6:20 ng/mL], vascular endothelial growth factor [VEGF: 50 ng/mL], and Flt-3 [100 ng/mL] (Peprotech; Rocky Hill, N.J.). The L−S+C+ cell population is heterogenous, but enriched for vasculogenic PCs (Tepper O M, Carr J, Allen R J, Jr., Chang C C, Lin C D, Tanaka R, Gupta S M, Levine J P, Saadeh P B, Warren S M: Decreased circulating progenitor cell number and failed mechanisms of stromal cell-derived factor-1 alpha mediated bone marrow mobilization impair diabetic tissue repair. Diabetes 2010; 59:1974-1983, the contents of which are hereby incorporated by reference in its entirety). Supplemented StemSpan is considered vasculogenic PC growth medium. All assays are performed on primary cultured PCs following 7 days of expansion.

Chemotaxis Assay: PC migration is measured using a modified Boyden chamber assay as previously described. Briefly, SDF-1α (100 ng/mL), PDGF-BB (100 ng/mL) or FBS (control) in vasculogenic PC growth medium or standard cell growth media is placed in the bottom of a 24-well plate. Cells (5×104)±AMD3100 (5-50 ng/mL)±rhPTH (5-50 ng/mL) is seeded onto fibronectin-coated (5 μg/cm2) transwell inserts. After 20 hours cells are harvested from the bottom chambers, washed, and centrifuged. Cell pellets are frozen at −80° C. Frozen cells are resuspended in CyQuant Green Fluorescent dye (Invitrogen) and the relative fluorescence is measured using a Synergy™ HT microplate reader (BioTek; Winooski, Vt.).

Adhesion Assay: Adhesion of PCs is measured in AMD3100 (5-50 ng/mL)±rhPTH (5-50 ng/mL). PCs (1×105 cells/chamber) are added to 4 well chamber slides (Fisher Scientific; Pittsburgh, Pa.) coated with fibronectin (5 μg/cm2) (Sigma) and incubated at 37° C. for 2 hours. Following incubation, non-adherent cells are removed before adherent cells are fixed with 1% paraformaldehyde. Adherent cells are stained with DAPI (4′,6 diamidino-2-phenylindole) (VectaShield; Vector Laboratories, Burlingame, Calif.) and viewed on an Olympus BX51 epifluorescent microscope. Adobe Photoshop CS3 (Adobe Systems; San Jose, Calif.) is used to quantify the number of cells/random high-powered field (hpf) under 100× magnification.

Proliferation Assay: Proliferation of PCs is measured using BrdU (5-Bromo-2′ deoxyuridine) labeling and fluorescent detection (Synergy™ HT microplate reader: BioTek; Winooski, Vt.). Proliferation is compared in media containing AMD3100 (5-50 ng/mL)±rhPTH (5-50 ng/mL).

Histology and Immunofluorescence: Fat cells are harvested on days 14, 21, and 28 for analysis. Frozen sections are stained with rat anti-mouse CD31 (PECAM; BD Biosciences) primary antibody and goat anti-rat IgG secondary (Alexafluor 594; Invitrogen). Control samples are prepared without primary antibody. Slides are mounted with DAPI (Sigma) and viewed on an Olympus BX51 epifluorescent microscope. DAPI is used to determine the sample outline; whereas, immunofluorescent CD31 staining is used to identify vascular structures (red staining) within the sample. Dual filter images are superimposed to illustrate wound architecture and vascular staining. Adobe Photoshop CS3 is used to segment and quantify positive CD31 staining. The vascular density of mouse wounds is determined by quantifying the total area of CD31+ staining (red) per megapixel (1×106 pixels square area) of wound stained. Paraffin sections are stained with hematoxylin and eosin (H&E) to compare wound architecture between treatment groups as well as to confirm the full-thickness nature of the punch biopsies.

Statistical Analysis: Data is presented as mean standard error of the mean. A one way ANOVA with post-hoc Tukey Kramer is used for comparison of wound closure rates, cPC number, and vascular staining between all groups studied. A Student's t test is used for comparison between groups for the functional assays. Statistical significance is considered to be p<0.05. The number of mice per treatment group is determined using G*Power (G*Power®, Melbourne, Australia) to provide a power greater than 0.80.

### Discussion

Our evidence suggests that neovascularization plays a significant role in tissue healing. The present application, encompasses, in part, an endogenous strategy to improve fat graft healing.

We show that endogenously mobilizing stem cells and concomitantly enhancing their trafficking yields a remarkable increase in healing. While systemic AMD3100 administration resulted in 59.7% bony ingrowth and PTH alone resulted in 56% bony ingrowth, together a synergistic effect of 90.6% bony regeneration was achieved; this was associated with significantly increased numbers of cPCs and CD31 staining in the trephine defect. We expect the same result with fat tissues. Our results suggest that mobilized vasculogenic PCs increase new blood vessel formation at the site of injury and substantially increase regeneration. Since the effect of combining AMD3100 and rhPTH was synergistic, it was clear that rhPTH was not just acting through a local proliferative effect, but was effectively improving PC trafficking. Our in vitro adhesion assay results strongly support that the nature of systemic rhPTH synergistic effect was through improved cPCs trafficking and tubule formation.

These findings reinforce that tissue healing is multifactorial. The present inventors have found that the combination of teriparatide (1-34 portion of PTH) and AMD3100 (PLERIXAFOR®) will enhance the healing at sites of fat harvesting and/or fat grafting. Without wishing to be bound by theory, targeting two completely different pathways, both equally essential to fat tissue growth, we will provide a level of healing not demonstrated before. Further, a major advantage of using these two drugs, aside from their biological efficacy, is that their safety in humans has already been established.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

1. A method of promoting healing at a site of injury, comprising on or more of the group of osteointegration and associated soft tissue, fat grafting injury and endochondral bone injury or disease, comprising the steps:
a. administering an effective amount of a bone marrow (BM)-derived vasculogenic progenitor cell mobilization factor to an animal or human having a site of injury;
b. administering an effective amount of a progenitor cell sensitizing factor to mobilize progenitor cells and sensitize said progenitor cells to one or more chemotactic agents present at the site of the injury.

2. The method of claim 1 wherein said mobilization factor is selected from the group consisting of CXCR4 agonist and partial agonists, granulocyte stimulating factor (G-CSF), granulocyte-macrophage stimulating factor (GM-CFS), Interleukin-1 (Il-1), Interleukin-3 (Il-3), interleukin-8 (Il-8), PIXY-321 (GM-CSF/Il-3 fusion protein), macrophage inflammatory protein, and growth related oncogene and agents and factors that modify the expression of the above factors.

3. The method of claim 2 wherein said CXCR4 agonists and partial agonists is AMD3100.

4. The method of claim 1 wherein said sensitizing factor is selected from the group consisting of parathyroid hormone and subunits of such hormone, NEL-like molecule-1, calreticulin, and closely related molecules, and agents and factors that modify the expression of the above factors.

5. The method of claim 4 wherein said parathyroid hormone and subunits thereof is recombinant human parathyroid hormone.

6. The method of claim 1 further comprising the step of administering at least one chemotactic factor to the area of the site of injury.

7. The method of claim 6 wherein the chemotactic agent is selected from the group consisting of stromal cell derived factors, transforming growth factors, bone morphogenic proteins, fibroblast growth factors, vascular endothelial growth factors, insulin-like growth factors, nerve growth factors, myostatins, platelet derived growth factors, neurotrophins, epidermal growth factors, keratinocyte growth factors, stem cell factors, thrombopoietins, Wnt signaling proteins, hypoxia inducible factors and agents capable of modifying the expression of one or more of the above factors.

8. The method of claim 1 wherein said mobilization factor and sensitization factor are administered in by subcutaneous, intraperitoneal or intravenous injection.

9. The method of claim 6 wherein said chemotactic agent is administered by injection.

10. The method of claim 6 wherein said chemotactic agent is administered by combining with the fat cells forming a fat graft.

11. The method of claim 1 wherein said injury is osteointegration of an implant and associated soft tissue.

12. The method of claim 1 wherein said injury is a fat grafting injury.

13. The method of claim 1 wherein said injury is an endochondral bone injury.

14. As article of manufacture, a therapeutic dosage form comprising effective amount of a bone marrow (BM)-derived vasculogenic progenitor cell mobilization factor and an effective amount of a progenitor cell sensitizing factor to mobilize progenitor cells and sensitize said progenitor cells to one or more chemotactic agents present at the site of injury comprising one or more of the group comprising osteointegration and associated soft tissue, fat graft injury and endochondral bone injury.

15. The dosage form of claim 14 wherein said effective amount of said mobilization factor and said effective amount of said sensitizing factor are lyophilized and held in a vial for reconstitution.

16. The dosage form of claim 14 wherein said effective amount of said mobilization factor and said effective amount of said sensitizing factor are held in a package with an effective amount of a chemotactic agent which chemotactic agent is administered to a site of injury to direct mobilized and sensitized progenitor cells to the site where healing is desired.

17. The dosage form of claim 16 wherein the chemotactic agent is selected from the group consisting of transforming growth factors, bone morphogenic proteins, fibroblast growth factors, vascular endothelial growth factors, stromal derived growth factors, insulin-like growth factors, nerve growth factors, myostatins, platelet derived growth factors, neurotrophins, epidermal growth factors, keratinocyte growth factors, stem cell factors, thrombopoietins, Wnt signaling proteins, hypoxia inducible factors and agents capable of modifying the expression of one or more of the above factors.

18. The dosage form of claim 16 wherein said chemotactic agent is lyophilized and held in a vial for reconstitution.

19. The dosage form of claim 16 wherein said chemotactic agent is administered by direct injection to soft tissue closely associated with the site of an implant.

20. The method of claim 16 wherein said chemotactic agent is administered by combining with the fat cells forming a fat graft.

21. The dosage form of claim 16 wherein said chemotactic agent is held in a sustained release vehicle.

22. The dosage form of claim 21 wherein said sustained release vehicle is a biopolymer.

23. The dosage form of claim 21 wherein said biopolymer is selected from the group comprising gelatin, polyglyconic and polylactic acid derivatives.

24. The dosage form of claim 23 wherein said biopolymer is formed as microspheres containing said chemotactic agent.

25. A kit for promoting healing at a site of injury, comprising on or more of the group of osteointegration and associated soft tissue, fat grafting injury and endochondral bone injury or disease, comprising the steps:
a. an effective amount of a bone marrow (BM)-derived vasculogenic progenitor cell mobilization factor to an animal or human having a site of injury;
b. an effective amount of a progenitor cell sensitizing factor to mobilize progenitor cells and sensitize said progenitor cells to one or more chemotactic agents present at the site of the injury; and,
c. instructions for their use to promote healing at a site of injury, comprising one or more of the group of osteointegration and associated soft tissue, fat grafting injury and endochondral bone injury or disease.

26. The kit of claim 24 wherein said bone marrow-derived vasculogenic cell mobilization factor is AMD3100.

27. The kit of claim 24 wherein said a progenitor cell sensitizing factor is selected from the group consisting of parathyroid hormone and subunits of such hormone.

28. The kit of claim 24 further comprising an implant.

29. The kit of claim 28 further comprising a chemotactic agent associated with the implant.

30. The kit of claim 24 further comprising a chemotactic agent for placement about the site of injury.

31. The kit of claim 24 further comprising a chemotactic agent for combining with fat cells used as a fat graft.
